(19) United States
(12) Patent Application Publication
(10) Pub. No.: US 2011/0053830 A1
(43) Pub. Date: Mar. 3, 2011

(54) METHODS FOR TREATING SEPTIC SHOCK

(75) Inventors: Steve Hefeneider, Portland, OR (US); Sharon L. McCoy, Portland, OR (US)

(73) Assignees: OREGON HEALTH & SCIENCE UNIVERSITY; THE GOVERNMENT OF THE UNITED STATES OF AMERICA, dba THE DEPARTMENT OF VETERANS AFFAIRS

(21) Appl. No.: 12/874,106

(22) Filed: Sep. 1, 2010

Related U.S. Application Data
(63) Continuation of application No. 11/834,506, filed on Aug. 6, 2007, now abandoned.
(60) Provisional application No. 60/836,172, filed on Aug. 7, 2006.

(51) Int. Cl. A61K 38/00; A61P 31/00; A61P 43/00

(52) U.S. Cl. 514/1.4

(57) ABSTRACT

Methods for the treatment of septic shock are disclosed herein. The methods include the use of a therapeutically effective amount of inhibitory peptides that inhibit TLR activity. The peptides can be used with other agents for the treatment of septic shock. In one embodiment, a therapeutically effective amount of a peptide is administered to a subject with septic shock, such as septic shock induced by an infection with gram negative bacteria.

### PRIORITY CLAIM

This application is a continuation of U.S. patent application Ser. No. 11/834,506, filed on Aug. 6, 2007, which in turn claims the benefit of U.S. provisional application 60/836,172, filed on Aug. 7, 2006. Both of the prior applications are incorporated herein by reference in their entirety.

### STATEMENT OF GOVERNMENT SUPPORT

This invention was made with United States government support pursuant to grant NIH-NIAID-AI065000, from the National Institutes of Health; the United States government has certain rights in the invention.

### FIELD

This relates to the field of septic shock, specifically to peptides for the treatment and/or prevention of septic shock, such as septic shock induced by infection with a gram negative bacteria.

### BACKGROUND

Septic shock (also known as sepsis) causes more than 150,000 deaths annually in the United States. Sepsis is defined as a clinical disorder whose symptoms may include well defined abnormalities in body temperature, heart rate, breathing rate, white blood cell count, hypotension, organ perfusion abnormalities, and multiple organ dysfunction. There are several causes of sepsis including bacterial (either gram negative or gram positive), fungal and viral infections, as well as non-infective stimuli such as multiple trauma, severe burns, organ transplantation and pancreatitis.

Septic patients usually die as a result of poor tissue perfusion and injury followed by multiple organ failure. It is well recognized that many of the responses that occur during septic shock are initiated by bacterial endotoxin (also known as lipoprotein or “LPS”) present on the surface of gram negative bacteria. This endotoxin is released upon the death or multiplication of the bacteria and is known to activate monocytes/macrophages or endothelial cells causing them to produce various mediator molecules such as toxic oxygen radicals, hydrogen peroxide, tumor neurosis factor-alpha (TNFa), and interleukin (IL-1, IL-6, and IL-8). These cellular and humoral inflammatory mediators evoke septic shock with symptoms ranging from chills and fever to circulatory failure, multiorgan failure, and death.

The profound effects of LPS are caused by the activation of LPS-sensitive cells, resulting in the excessive release of cytokines and other inflammatory mediators. Recent work has shown that Toll-like receptor (TLR)-4 plays a key role in LPS recognition (Poltorak et al., Science 282:2085-8, 1998). The TLRs are elements of the innate immune system that function both as receptors for pathogens and initiators of intracellular signaling, leading to an inflammatory response. TLRs have a conserved amino acid region in their cytoplasmic tails defined as the Toll/IL-1R (TIR) domain (Akira and Takeda, Nat Rev Immunol 4:499-511, 2004). After ligand binding, TLRs undergo conformational changes required for the recruitment of downstream signaling molecules. These include the adaptor molecule myeloid differentiation primary-response protein 88 (MyD88), IL-1R associated kinases (IKAKs), and tumor-necrosis factor-receptor-associated factor 6 (TRAF6). This pathway culminates in the translocation of nuclear factor-KB (NF-κB) to the nucleus and the production of proinflammatory cytokines, chemokines, inducible nitric oxide synthase (iNOS) and up-regulation of cell adhesion molecules, leading to an inflammatory host immune response (Akira and Takeda, Nat Rev Immunol 4:499-511, 2004).

A need remains for effective treatments for septic shock, such as the septic shock induced by gram negative bacteria. These treatments can include the administration of pharmaceutical compositions including polypeptides.

### SUMMARY

Methods are disclosed herein for the treatment of septic shock. The methods utilize peptides that inhibit toll-like receptor activity.

In several embodiments, these methods utilize a therapeutically effective amount of a fusion protein that includes or consists of a first polypeptide and a second polypeptide. The first polypeptide consists of 11 to 18 consecutive amino acids of the amino acid sequence set forth as SEQ ID NO: 1 (A52R), or 11 to 18 amino acids of the amino acid sequence set forth as SEQ ID NO: 1 with a single amino acid substitution that inhibit TLR activity with the same or greater affinity as the 11 to 18 consecutive amino acids of the amino acid sequence set forth as SEQ ID NO: 1. The second polypeptide consists of seven to ten consecutive arginine residues. In some examples, the subject has septic shock induced by infection with gram negative bacteria. Additional agents can be administered to the subject, such as anti-microbial agents.

The foregoing and other features and advantages will become more apparent from the following detailed description of several embodiments, which proceeds with reference to the accompanying figures.

### BRIEF DESCRIPTION OF THE FIGURES

FIG. 1 is a set of bar graphs showing P13 inhibition of macrophage inflammatory protein (MIP)-2 and interleukin (IL)-6. Murine endothelial cell line bEND.3 (2×10⁵ cells/48-well plate) were pre-treated for 15 minutes with P13 or scrambled control peptide at matching peptide concentrations (15 or 20 μg/well) and then with LPS (12.5 ng/well). MIP-2 and IL-6 were quantified by ELISA from 4-hour cell-free supernatants. Cytokine values represent means+/−S.D. from quadruplicate wells/experimental condition.

FIG. 2 is a set of bar graphs showing P13 inhibition of serum inflammatory mediators. BALB/c mice were injected intraperitoneally (i.p.) with 5 mg/kg LPS and 30 minutes later injected i.p. with P13 (15, 50, or 75 μg) or PBS. Two hours after LPS injection, serum was collected and TNF-α and soluble ICAM-1 were quantified by ELISA. Values represent means+/−S.D.

FIG. 3 is a line graph illustrating decreased mortality in P13-treated mice after high dose LPS. BALB/c mice were injected i.p. with LPS (70 mg/kg) and 2 and 6 hrs later treated s.c. with P13 or control peptide (75 μg/mouse/injection). The mice (10 animals/group) were monitored every 3-4 hours for lethality, and the experiment was terminated after 96 hrs.

FIG. 4 is the amino acid sequence of several A52R (SEQ ID NO: 1) polypeptides. Each peptide sequence is consecutive amino acids from A52R (SEQ ID NO: 1).

### SEQUENCE LISTING

The nucleic and amino acid sequences listed in the accompanying sequence listing are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. The Sequence Listing is submitted as an ASCII text file [name of file 6915-79180-06_Sequence_Listing.txt, date of creation Aug. 28, 2010, size of ASCII text file in bytes 6.78 KB], which is incorporated by reference herein.

In the accompanying sequence listing:

SEQ ID NO: 1 is the amino acid sequence of A52R.

SEQ ID NO: 2 is an exemplary nucleic acid sequence encoding SEQ ID NO: 1.

SEQ ID NO: 3 is the amino acid sequence of P13.

SEQ ID NO: 4 is the amino acid sequence of a control peptide.

SEQ ID NO: 5 is the amino acid sequence of P7.

SEQ ID NOs: 6-21 are the amino acid sequences of A52R polypeptides.

### DETAILED DESCRIPTION

Methods for the treatment of septic shock are disclosed herein. The methods include the use of a therapeutically effective amount of inhibitory peptides that inhibits Toll-like receptor activity. The peptides can be used with other agents for the treatment of septic shock. In several embodiments, the septic shock is caused by an infection with a gram negative bacteria.

### Terms

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

In order to facilitate review of the various embodiments of this disclosure, the following explanations of specific terms are provided:

Animal: Living multi-cellular vertebrate organisms, a category that includes, for example, mammals and birds. The term mammal includes both human and non-human mammals. Similarly, the term “subject” includes both human and veterinary subjects.

cDNA (complementary DNA): A piece of DNA lacking internal, non-coding segments (introns) and regulatory sequences that determine transcription. cDNA is synthesized in the laboratory by reverse transcription from messenger RNA extracted from cells.

Cell Adhesion Molecules (CAMs): Proteins located on the cell surface involved with the binding with other cells or with the extracellular matrix (ECM) in cell adhesion. These proteins are typically transmembrane receptors and are composed of three domains: an intracellular domain that interacts with the cytoskeleton, a transmembrane domain and an extracellular domain that interacts either with other CAMs of the same kind (homophilic binding) or with other CAMs or the extracellular matrix (heterophilic binding). Intracellular adhesion molecule (ICAM)-1 is a cellular adhesion molecule.

Conservative variants: “Conservative” amino acid substitutions are those substitutions that do not substantially affect or decrease an activity of a polypeptide, such as the ability to bind Toll-like receptor (TLR)-3, decrease serum TNF-α, decrease the production of MIP-2, decrease the production of IL-2, and/or reduce ICAM levels. Specific, non-limiting examples of a conservative substitution include the following examples:

| Original Residue | Conservative Substitutions |
|---|---|
| Al | Ser |
| Arg | Lys |
| Asn | Gln, His |
| Asp | Glu |
| Cys | Ser |
| Gln | Asn |
| Glu | Asp |
| His | Asn; Gln |
| Ile | Leu, Val |
| Leu | Ile; Val |
| Lys | Arg; Gln; Glu |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

The term conservative variation also includes the use of a substituted amino acid in place of an unsubstituted parent amino acid, provided that the polypeptide binds to TLR-3 with the same affinity as the unsubstituted (parental) polypeptide. Non-conservative substitutions are those that reduce the ability of the polypeptide to bind TLR-3.

Consists Essentially Of/Consists Of: With regard to a polypeptide, a polypeptide that consists essentially of a specified amino acid sequence if it does not include any additional amino acid residues. However, the polypeptide can include additional non-peptide components, such as labels (for example, fluorescent, radioactive, or solid particle labels), sugars or lipids. With regard to a polypeptide, a polypeptide that consists of a specified amino acid sequence does not include any additional amino acid residues, nor does it include additional non-peptide components, such as lipids, sugars or labels.

Cytokine: The term “cytokine” is used as a generic name for a diverse group of soluble proteins and peptides that act as humoral regulators at nano- to picomolar concentrations and which, either under normal or pathological conditions, modulate the functional activities of individual cells and tissues. These proteins also mediate interactions between cells directly and regulate processes taking place in the extracellular environment. Examples of cytokines include, but are not limited to, tumor necrosis factor α (TNFα), interleukin 6 (IL-6), interleukin 10 (IL-10), interleukin 12 (IL-12), macrophage inflammatory protein 2 (MIP-2), KC, and interferon-γ (INF-γ).

Degenerate variant: A polynucleotide encoding a peptide, such as an A52R peptide, that includes a sequence that is degenerate as a result of the genetic code. There are 20 natural amino acids, most of which are specified by more than one codon. Therefore, all degenerate nucleotide sequences are included in this disclosure as long as the amino acid sequence of the polypeptide encoded by the nucleotide sequence is unchanged.

Expression Control Sequences: Nucleic acid sequences that regulate the expression of a heterologous nucleic acid sequence to which it is operatively linked. Expression control sequences are operatively linked to a nucleic acid sequence when the expression control sequences control and regulate the transcription and, as appropriate, translation of the nucleic acid sequence. Thus, expression control sequences can include appropriate promoters, enhancers, transcription terminators, a start codon (i.e., ATG) in front of a protein-encoding gene, splicing signal for introns, maintenance of the correct reading frame of that gene to permit proper translation of mRNA, and stop codons. The term “control sequences” is intended to include, at a minimum, components whose presence can influence expression, and can also include additional components whose presence is advantageous, for example, leader sequences and fusion partner sequences. Expression control sequences can include a promoter.

A promoter is a minimal sequence sufficient to direct transcription. Also included are those promoter elements which are sufficient to render promoter-dependent gene expression controllable for cell-type specific, tissue-specific, or inducible by external signals or agents; such elements may be located in the 5′ or 3′ regions of the gene. Both constitutive and inducible promoters are included (see e.g., Bitter et al., Methods in Enzymology 153:516-544, 1987). For example, when cloning in bacterial systems, inducible promoters such as pL of bacteriophage lambda, plac, ptrp, ptac (ptrp-lac hybrid promoter) and the like can be used. In one embodiment, when cloning in mammalian cell systems, promoters derived from the genome of mammalian cells (such as the metallothionein promoter) or from mammalian viruses (such as the retrovirus long terminal repeat; the adenovirus late promoter; the vaccinia virus 7.5K promoter) can be used. Promoters produced by recombinant DNA or synthetic techniques can also be used to provide for transcription of the nucleic acid sequences.

Heterologous: Originating from separate genetic sources or species. A polypeptide that is heterologous to a vaccinia virus protein, such as A52R, originates from a nucleic acid that does not encode this polypeptide. In one specific, non-limiting example, a polypeptide comprising eleven consecutive amino acids from A52R, or at most twelve consecutive amino acids from A52R, and a heterologous amino acid sequence includes a β-galactosidase, a maltose binding protein, and albumin, hepatitis B surface antigen, a series of tyrosines, or a signal peptide. Generally, an antibody that specifically binds to a protein of interest will not specifically bind to a heterologous protein.

Host cells: Cells in which a vector can be propagated and its DNA expressed. The cell may be prokaryotic or eukaryotic. The cell can be mammalian, such as a human cell. The term also includes any progeny of the subject host cell. It is understood that all progeny may not be identical to the parental cell since there may be mutations that occur during replication. However, such progeny are included when the term “host cell” is used.

Immune response: A response of a cell of the immune system, such as a B cell, T cell, or monocyte, to a stimulus. In one embodiment, the response is specific for a particular antigen (an “antigen-specific response”). In one embodiment, an immune response is a T cell response, such as a CD4+ response or a CD8+ response. In another embodiment, the response is a B cell response, and results in the production of specific antibodies.

Inhibiting or treating a disease: Inhibiting a disease, such as septic shock, refers to inhibiting the full development of a disease. In several examples, inhibiting a disease refers to lessening symptoms of septic shock, such as preventing the development of multi-organ failure or circulatory failure in a person who is known to be septic, or lessening a sign or symptom of septic shock, such as fever. “Treatment” refers to a therapeutic intervention that ameliorates a sign or symptom of a disease or pathological condition related to the disease, such as septic shock, such as reducing fever or stabilizing blood pressure in a subject with septic shock.

Isolated: An “isolated” biological component (such as a nucleic acid or protein or organelle) has been substantially separated or purified away from other biological components in the cell of the organism in which the component naturally occurs, i.e., other chromosomal and extra-chromosomal DNA and RNA, proteins and organelles. Nucleic acids and proteins that have been “isolated” include nucleic acids and proteins purified by standard purification methods. The term also embraces nucleic acids and proteins prepared by recombinant expression in a host cell as well as chemically synthesized nucleic acids.

Label: A detectable compound or composition that is conjugated directly or indirectly to another molecule to facilitate detection of that molecule. Specific, non-limiting examples of labels include fluorescent tags, enzymatic linkages, and radioactive isotopes.

Linker sequence: A linker sequence is an amino acid sequence that covalently links two polypeptide domains. Linker sequences can be included in the between an A52R peptide and a number of tyrosines as disclosed herein to provide rotational freedom to the linked polypeptide domains. By way of example, in a recombinant polypeptide comprising two domains, linker sequences can be provided between them. Linker sequences, which are generally between 2 and 25 amino acids in length, are well known in the art and include, but are not limited to, the glycine(4)-serine spacer (GGGGS ×3) described by Chaudhary et al., Nature 339:394-397, 1989.

Lymphocytes: A type of white blood cell that is involved in the immune defenses of the body. There are two main types of lymphocytes: B cells and T cells.

Mammal: This term includes both human and non-human mammals. Similarly, the term “subject” includes both human and veterinary subjects.

Oligonucleotide: A linear polynucleotide sequence of up to about 100 nucleotide bases in length.

Open reading frame (ORF): A series of nucleotide triplets (codons) coding for amino acids without any internal termination codons. These sequences are usually translatable into a peptide.

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence, such as a sequence that encodes an A52R polypeptide. Generally, operably linked DNA sequences are contiguous and, where necessary to join two protein-coding regions, in the same reading frame.

Peptide Modifications: The disclosed peptides include synthetic embodiments of peptides described herein. In addition, analogs (non-peptide organic molecules), derivatives (chemically functionalized peptide molecules obtained starting with the disclosed peptide sequences) and variants (homologs) of these proteins can be utilized in the methods described herein. Each polypeptide of this disclosure is comprised of a sequence of amino acids, which may be either L- and/or D-amino acids, naturally occurring and otherwise.

Peptides can be modified by a variety of chemical techniques to produce derivatives having essentially the same activity as the unmodified peptides, and optionally having other desirable properties. For example, carboxylic acid groups of the protein, whether carboxyl-terminal or side chain, can be provided in the form of a salt of a pharmaceutically-acceptable cation or esterified to form a C₁-C₁₆ ester, or converted to an amide of formula N1R₁R₂ wherein R₁ and R₂ are each independently H or C₁-C₁₆ alkyl, or combined to form a heterocyclic ring, such as a 5- or 6-membered ring. Amino groups of the peptide, whether amino-terminal or side chain, can be in the form of a pharmaceutically-acceptable acid addition salt, such as the HCl, HBr, acetic, benzoic, toluene sulfonic, maleic, tartaric and other organic salts, or can be modified to C₁-C₁₆ alkyl or dialkyl amino or further converted to an amide.

Hydroxyl groups of the peptide side chains may be converted to C₁-C₁₆ alkoxy or to a C₁-C₁₆ ester using well-recognized techniques. Phenyl and phenolic rings of the peptide side chains may be substituted with one or more halogen atoms, such as fluorine, chlorine, bromine or iodine, or with C₁-C₁₆ alkyl, C₁-C₁₆ alkoxy, carboxylic acids and esters thereof, or amides of such carboxylic acids. Methylene groups of the peptide side chains can be extended to homologous C₂-C₄ alkylenes. Thiols can be protected with any one of a number of well-recognized protecting groups, such as acetamide groups. Those skilled in the art will also recognize methods for introducing cyclic structures into the peptides of this invention to select and provide conformational constraints to the structure that result in enhanced stability.

Peptidomimetic and organomimetic embodiments are envisioned, whereby the three-dimensional arrangement of the chemical constituents of such peptido- and organomimetics mimic the three-dimensional arrangement of the peptide backbone and component amino acid side chains, resulting in such peptido- and organomimetics of a polypeptide having measurable or enhanced ability to generate an immune response. For computer modeling applications, a pharmacophore is an idealized three-dimensional definition of the structural requirements for biological activity. Peptido- and organomimetics can be designed to fit each pharmacophore with current computer modeling software (using computer assisted drug design or CADD). See Walters, “Computer-Assisted Modeling of Drugs,” in Klegerman & Groves, eds., 1993, Pharmaceutical Biotechnology, Interpharm Press: Buffalo Grove, Ill., pp. 165-174 and Principles of Pharmacology, Munson (ed.) 1995, Ch. 102, for descriptions of techniques used in CADD. Also included are mimetics prepared using such techniques.

Pharmaceutically acceptable carriers: The pharmaceutically acceptable carriers of use are conventional. Remington's Pharmaceutical Sciences, by E. W. Martin, Mack Publishing Co., Easton, Pa., 15th Edition (1975), describes compositions and formulations suitable for pharmaceutical delivery of the fusion proteins herein disclosed.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (such as powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

A “therapeutically effective amount” is a quantity of a composition or a cell to achieve a desired effect in a subject being treated. For instance, this can be the amount necessary to inhibit septic shock, reduce fever, or prevent multi-organ failure in a subject infected with a gram negative bacteria. When administered to a subject, a dosage will generally be used that will achieve target tissue concentrations that has been shown to achieve an in vitro effect.

Polynucleotide: The term polynucleotide or nucleic acid sequence refers to a polymeric form of nucleotide at least 10 bases in length. A recombinant polynucleotide includes a polynucleotide that is not immediately contiguous with both of the coding sequences with which it is immediately contiguous (one on the 5′ end and one on the 3′ end) in the naturally occurring genome of the organism from which it is derived. The term therefore includes, for example, a recombinant DNA which is incorporated into a vector; into an autonomously replicating plasmid or virus; or into the genomic DNA of a prokaryote or eukaryote, or which exists as a separate molecule (e.g., a cDNA) independent of other sequences. The nucleotides can be ribonucleotides, deoxyribonucleotides, or modified forms of either nucleotide. The term includes single- and double-stranded forms of DNA.

Polypeptide: Any chain of amino acids, regardless of length or post-translational modification (e.g., glycosylation or phosphorylation). In one embodiment, the polypeptide is an A52R polypeptide. A polypeptide can be between 5 and 25 amino acids in length. In one embodiment, a polypeptide is from about 10 to about 20 amino acids in length. In yet another embodiment, a polypeptide is from about 11 to about 18 amino acids in length. With regard to polypeptides, the word “about” indicates integer amounts. Thus, in one example, a polypeptide “about” 11 amino acids in length is from 10 to 12 amino acids in length. Similarly, a polypeptide “about” 18 amino acids in length is from about 17 to about 19 amino acids in length. Thus, a polypeptide “about” a specified number of residues can be one amino acid shorter or one amino acid longer than the specified number. A fusion polypeptide includes the amino acid sequence of a first polypeptide and a second different polypeptide (for example, a heterologous polypeptide), and can be synthesized as a single amino acid sequence. In one example a fusion polypeptide includes an A52R polypeptide and a series of arginines, such as 8 to 11 arginines.

Probes and primers: A probe comprises an isolated nucleic acid attached to a detectable label or reporter molecule. Primers are short nucleic acids, preferably DNA oligonucleotides, of about 15 nucleotides or more in length. Primers may be annealed to a complementary target DNA strand by nucleic acid hybridization to form a hybrid between the primer and the target DNA strand, and then extended along the target DNA strand by a DNA polymerase enzyme. Primer pairs can be used for amplification of a nucleic acid sequence, for example by polymerase chain reaction (PCR) or other nucleic-acid amplification methods known in the art. One of skill in the art will appreciate that the specificity of a particular probe or primer increases with its length. Thus, for example, a primer comprising 20 consecutive nucleotides will anneal to a target with a higher specificity than a corresponding primer of only 15 nucleotides. Thus, in order to obtain greater specificity, probes and primers can be selected that comprise about 20, 25, 30, 35, 40, 50 or more consecutive nucleotides.

Purified: The polypeptides disclosed herein can be purified (and/or synthesized) by any of the means known in the art (see, e.g., Guide to Protein Purification, ed. Deutscher, Meth. Enzymol. 185, Academic Press, San Diego, 1990; and Scopes, Protein Purification: Principles and Practice, Springer Verlag, New York, 1982). Substantial purification denotes purification from other proteins or cellular components. A substantially purified protein is at least about 60%, 70%, 80%, 90%, 95%, 98% or 99% pure. Thus, in one specific, non-limiting example, a substantially purified protein is 90% free of other proteins or cellular components. Thus, the term purified does not require absolute purity; rather, it is intended as a relative term. For example, a purified nucleic acid is one in which the nucleic acid is more enriched than the nucleic acid in its natural environment within a cell. In additional embodiments, a nucleic acid or cell preparation is purified such that the nucleic acid or cell represents at least about 60% (such as, but not limited to, 70%, 80%, 90%, 95%, 98% or 99%) of the total nucleic acid or cell content of the preparation, respectively.

Recombinant: A recombinant nucleic acid is one that has a sequence that is not naturally occurring or has a sequence that is made by an artificial combination of two otherwise separated segments of sequence. This artificial combination is often accomplished by chemical synthesis or, more commonly, by the artificial manipulation of isolated segments of nucleic acids, e.g., by genetic engineering techniques. A recombinant polypeptide has an amino acid sequence that is not naturally occurring or that is made by two otherwise separated segments of an amino acid sequence.

Selectively hybridize: Hybridization under moderately or highly stringent conditions that excludes non-related nucleotide sequences.

In nucleic acid hybridization reactions, the conditions used to achieve a particular level of stringency will vary, depending on the nature of the nucleic acids being hybridized. For example, the length, degree of complementarity, nucleotide sequence composition (for example, GC v. AT content), and nucleic acid type (for example, RNA versus DNA) of the hybridizing regions of the nucleic acids can be considered in selecting hybridization conditions. An additional consideration is whether one of the nucleic acids is immobilized, for example, on a filter.

A specific example of progressively higher stringency conditions is as follows: 2×SSC/0.1% SDS at about room temperature (hybridization conditions); 0.2×SSC/0.1% SDS at about room temperature (low stringency conditions); 0.2×SSC/0.1% SDS at about 42° C. (moderate stringency conditions); and 0.1×SSC at about 68° C. (high stringency conditions). One of skill in the art can readily determine variations on these conditions (e.g., Molecular Cloning: A Laboratory Manual, 2nd ed., vol. 1-3, ed. Sambrook et al., Cold Spring Harbor Laboratory Press, Cold Spring Harbor, N.Y., 1989). Washing can be carried out using only one of these conditions, e.g., high stringency conditions, or each of the conditions can be used, e.g., for 10-15 minutes each, in the order listed above, repeating any or all of the steps listed. However, as mentioned above, optimal conditions will vary, depending on the particular hybridization reaction involved, and can be determined empirically.

Sequence identity: The similarity between amino acid sequences is expressed in terms of the similarity between the sequences, otherwise referred to as sequence identity. Sequence identity is frequently measured in terms of percentage identity (or similarity or homology); the higher the percentage, the more similar the two sequences are. Homologs or variants of a polypeptide will possess a relatively high degree of sequence identity when aligned using standard methods.

Methods of alignment of sequences for comparison are well known in the art. Various programs and alignment algorithms are described in: Smith and Waterman, Adv. Appl. Math. 2:482, 1981; Needleman and Wunsch, J. Mol. Biol. 48:443, 1970; Higgins and Sharp, Gene 73:237, 1988; Higgins and Sharp, CABIOS 5:151, 1989; Corpet et al., Nucleic Acids Research 16:10881, 1988; and Pearson and Lipman, Proc. Natl. Acad. Sci. USA 85:2444, 1988. Altschul et al., Nature Genet. 6:119, 1994, presents a detailed consideration of sequence alignment methods and homology calculations.

The NCBI Basic Local Alignment Search Tool (BLAST) (Altschul et al., J. Mol. Biol. 215:403, 1990) is available from several sources, including the National Center for Biotechnology Information (NCBI, Bethesda, Md.) and on the internet, for use in connection with the sequence analysis programs blastp, blastn, blastx, tblastn and tblastx. A description of how to determine sequence identity using this program is available on the NCBI website on the internet.

Homologs and variants of a polypeptide are typically characterized by possession of at least 75%, for example at least 80%, sequence identity counted over the full length alignment with the amino acid sequence of the A52R polypeptide using the NCBI Blast 2.0, gapped blastp set to default parameters. For comparisons of amino acid sequences of greater than about 30 amino acids, the Blast 2 sequences function is employed using the default BLOSUM62 matrix set to default parameters, (gap existence cost of 11, and a per residue gap cost of 1). When aligning short peptides (fewer than around 30 amino acids), the alignment should be performed using the Blast 2 sequences function, employing the PAM30 matrix set to default parameters (open gap 9, extension gap 1 penalties). Proteins with even greater similarity to the reference sequences will show increasing percentage identities when assessed by this method, such as at least 80%, at least 85%, at least 90%, at least 95%, at least 98%, or at least 99% sequence identity. When less than the entire sequence is being compared for sequence identity, homologs and variants will typically possess at least 80% sequence identity over short windows of 10-20 amino acids, and can possess sequence identities of at least 85% or at least 90% or 95% depending on their similarity to the reference sequence. Methods for determining sequence identity over such short windows are available at the NCBI website on the internet. One of skill in the art will appreciate that these sequence identity ranges are provided for guidance only; it is entirely possible that strongly significant homologs could be obtained that fall outside of the ranges provided.

Septic shock or sepsis: A clinical disorder whose symptoms can include abnormalities in body temperature, heart rate, breathing rate, white blood cell count, hypertension, organ perfusion abnormalities, and multiple organ dysfunction. Septic shock can be caused by bacterial (either gram negative or gram positive), fungal, viral or other infection. Septic shock is commonly caused by “gram-negative” endotoxin-producing aerobic rods such as Escherichia coli, Klebsiella pneumoniae, Proteus species, Pseudomonas aeruginosa and Salmonella. Septic shock involved with gram negative bacteria is referred to as “endotoxic shock”. A significant portion of the peripheral responses occurring during septic shock are initiated by endotoxin (also referred to herein as lipopolysaccharide or “LPS”), an outer-membrane component of gram-negative bacteria which is released upon the death or multiplication of the bacteria. Without being bound by theory, the manner in which LPS evokes its effects is by binding to cells such as monocytes/macrophages or endothelial cells and triggering them to produce various mediators, such as oxygen radicals, hydrogen peroxide, tumor necrosis factor-alpha (TNF-.alpha.), and various interleukins (IL-1, IL-6, and IL-8). Gram-positive bacteria, particularly pneumococcal or streptococcal bacteria, can produce a similar clinical syndrome as endotoxic shock. Thus, as used herein, the term “septic shock” refers to septic shock involved with gram negative and/or gram positive bacteria.

Transduced: A transduced cell is a cell into which has been introduced a nucleic acid molecule by molecular biology techniques. As used herein, the term transduction encompasses all techniques by which a nucleic acid molecule might be introduced into such a cell, including transfection with viral vectors, transformation with plasmid vectors, and introduction of naked DNA by electroporation, lipofection, and particle gun acceleration.

Toll-like Receptors (TLR): Conserved molecular receptors that recognize bacterial, fungal, protozoal and viral components. In humans, at least ten known TLRs are known to recognize different pathogenic molecular markers, such as viral double-stranded RNA (TLR3), flagellin (TLR5) and components of bacterial cell wall including lipopolysaccharide (LPS; TLR4) or lipopeptide (TLR2). Ligand-stimulated TLRs interact with various Toll/interleukin-1 receptor (TIR) domain. The protein sequence for TLR3 can be found as GENBANK® Accession Nos. ABF06634 and ABF06637 (Apr. 30, 2006), which are incorporated by reference herein. The protein sequence for TLR4 can be found as GENBANK® Accession Nos. NM_{—}138554 (Jul. 30, 2007), herein incorporated by reference. Thirteen TLRs (TLR1 to TLR13) have been identified in humans and mice together, and equivalent forms of many of these have been found in other mammalian species.

TLRs recognize conserved motifs found in various pathogens and mediate defense responses. Triggering of the TLR pathway leads to the activation of NF-κB and subsequent regulation of immune and inflammatory genes. The TLRs and members of the interleukin (IL)-1 receptor family share a conserved stretch of about 200 amino acids known as the TIR domain. Upon activation, TLRs associate with a number of cytoplasmic adaptor proteins containing TIR domains including MyD88 (myeloid differentiation factor), MAL/TIRAP (MyD88-adaptor-like/TIR-associated protein), TRIF (Toll-receptor-associated activator of interferon) and TRAM (Toll-receptor associated molecule). Cells in vivo, express TLRs as 4- and 6-kb transcripts that are most abundant in placenta and pancreas. TLR activity includes activation of NF-κB. Activation of TLRs can result in increased production of tumor necrosis factor α (TNFα), interleukin (IL)-1β, IL-6, IL-8, IL-12, RANTES, MIP-1α, and MIP-1β.

Vector: A nucleic acid molecule as introduced into a host cell, thereby producing a transformed host cell. A vector may include nucleic acid sequences that permit it to replicate in a host cell, such as an origin of replication. A vector may also include one or more selectable marker gene and other genetic elements known in the art. Vectors include plasmid vectors, including plasmids for expression in gram negative and gram positive bacterial cell. Exemplary vectors include those for expression in E. coli and Salmonella. Vectors also include viral vectors, such as, but are not limited to, retrovirus, orthopox, avipox, fowlpox, capripox, suipox, adenoviral, herpes virus, alpha virus, baculovirus, Sindbis virus, vaccinia virus and poliovirus vectors. Vectors also include vectors for expression in yeast cells.

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below. The term “comprises” means “includes.” All publications, patent applications, patents, and other references mentioned herein are incorporated by reference in their entirety. In case of conflict, the present specification, including explanations of terms, will control. In addition, the materials, methods, and examples are illustrative only and not intended to be limiting.

### A 52R Polypeptides

Polypeptides are disclosed herein that include about 10 to about 18 amino acids of the A52R polypeptide of vaccinia virus, or a single amino acid substitution thereof that retains the ability to inhibit TLR activity compared to the parent polypeptide. Therapeutic polypeptides are disclosed herein that include, or consist of, two components: (a) a first polypeptide consisting of 11 to 18 consecutive amino acids of the amino acid sequence set forth as SEQ ID NO: 1 (A52R), or wherein the first polypeptide consists of 11 to 18 amino acids of amino acid sequence set forth as SEQ ID NO: 1 with a single amino acid substitution thereof that retains the ability to inhibit toll-like receptor (TLR) activity as the 11 to 18 consecutive amino acids of the amino acid sequence set forth as SEQ ID NO: 1, and (b) a second peptide consisting of seven to ten consecutive arginine residues. The second polypeptide can be covalently bound to the first polypeptide, such as in a fusion polypeptide (including both the second and first polypeptide sequences).

In one embodiment, A52R has the following amino acid sequence:

MDIKIDISIS GDKFTVTTRR ENEERKKYLP LQKEKTTDVI
KPDYLEYDDL LDRDEMFTIL EEYFMYRGLL GLRIKYGRLF
NEIKKFDNDA EEQFGTIEEL KQKLRLNSEE GADNFIDYIK
VQKQDIVKLT VYDCISMIGL CACVVDVWRN EKLFSRWKYC
LRAIKLFIND HMLDKIKSIL QNRLVYVEMS.
(SEQ ID NO: 1, see GENBANK ® Accession No.
ABP97440, incorporated herein by reference).

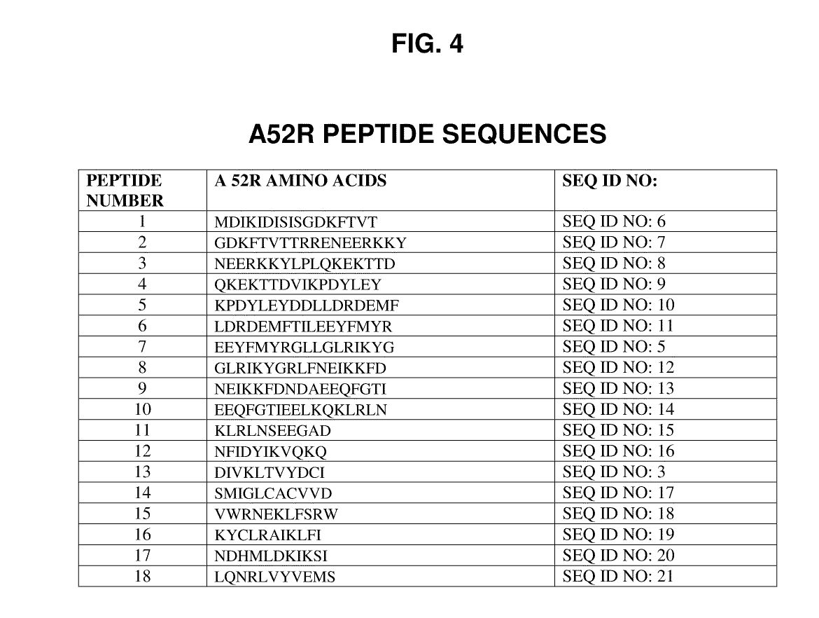

In one embodiment, the first polypeptide can include, or consist of, about 10, about 11, about 12, about 13, about 14, about 15 about 16, about 17 or about 18 consecutive amino acids of SEQ ID NO: 1. Exemplary polypeptides are shown in FIG. 4. In additional embodiments the polypeptide includes, or consists of, 10 to 18, 11 to 17, 11 to 16, 11 to 15, 11 to 14, 11 to 13, or 11 to 12 consecutive amino acids of SEQ ID NO: 1. In specific, non limiting examples, the polypeptide includes, or consists of, 11 to 12 consecutive amino acids of SEQ ID NO: 1. Specific non-limiting examples of a polypeptide of use include DIVKLTVYDCI (P13, amino acids 125 to 135 of SEQ ID NO: 1, also shown as SEQ ID NO: 3) or EEYFMYRGLLGLRIKYG (P7, amino acids 70 to 86 of SEQ ID NO: 1, also shown as SEQ ID NO: 5), see also FIG. 4.

In addition, the polypeptide can include, or consist of, a single amino acid substitution of the amino acid sequence set forth as DIVKLTVYDCI (amino acids 125 to 135 of SEQ ID NO: 1, amino acids 70 to 86 of SEQ ID NO: 1. In several examples, the amino acid at position 1, 2, 3, 4, 5, 6, 7, 8, 9, 10 or 11 of amino acid 125 to 135 is substituted with another amino acid, but the polypeptide retains the ability to inhibit TLR activity as compared to the parental polypeptide from SEQ ID NO: 1 (without any amino acid substitutions). Similarly, the amino acid of position 1, 2, 3, 4, 5, 6, 7, 8, 9, 10, 11, 12, 13, 14, 15, 16 or 17 of amino acids 70 to 86 can be substituted with another amino acid, but the polypeptide retains the ability to inhibit TLR activity.

In several specific, non-limiting examples, a peptide “retains the ability to inhibit TLR activity” if it retains the ability to inhibit the production of on or more of TNFα, IL-1β, IL-6, IL-8, IL-12, RANTES, MIP-1α or MIP-1β as a parental polypeptide. In one specific, non-limiting example, a peptide “retains the ability to inhibit TLR activity” if it retains the ability to inhibit the production of on or more of TNFα, IL-1β, IL-6, IL-8, IL-12, RANTES, MIP-1α or MIP-1β as compared to a parental polypeptide, if the production of TNFα, IL-1β, IL-6, IL-8, IL-12, RANTES, MIP-1α or MIP-1β does not differ statistically from the amount of produced by the parental polypeptide. Statistically assays are well known in the art and include, for example, a Student's T test.

The polypeptide disclosed herein can include seven to ten consecutive arginine residues covalently bound to the A52R polypeptide, such as in a fusion protein. Thus, the first polypeptide and the second polypeptide can be produced as a single amino acid sequence. In one specific, non-limiting example, the seven to ten arginines are at the amino terminal end, and the A52R polypeptide is at the carboxy terminal end of a single polypeptide sequence. In another specific, non-limiting example, the seven to ten arginines are at the carboxy terminal end, and the A52R polypeptide is at the amino terminal end of a single polypeptide sequence. The polypeptide can include 7, 8, 9 or 10 consecutive arginine residues. In several non-limiting examples, the polypeptide consists of 7 to 10 arginine residues covalently linked to 10 to 18 consecutive residues of SEQ ID NO: 1. In additional non-limiting examples, the polypeptide consists of 7 to 10 arginine residues covalently linked to 10 to 18 consecutive residues of SEQ ID NO: 1 with a single amino acid substitution that retains the ability to inhibit TLR activity as compared to the parental polypeptide. In a further non-limiting example, the polypeptide consists of, or consists essentially of, amino acids 70 to 86 of SEQ ID NO: 1 covalently linked to 9 arginine residues. In a further non-limiting example, the polypeptide consists of, or consists essentially of, amino acids 70 to 86 of SEQ ID NO: 1 with a single amino acid substitution covalently linked to 9 arginine residues. Additional polypeptides consist of, or consist essentially of, amino acids 125 to 135 of SEQ ID NO: 1, or a single substitution thereof, covalently linked to 9 arginine residues.

The polypeptides disclosed herein can be chemically synthesized by standard methods, or can be produced recombinantly. An exemplary process for polypeptide production is described in Lu et al., Federation of European Biochemical Societies Letters. 429:31-35, 1998. They can also be isolated by methods including preparative chromatography and immunological separations.

Polynucleotides encoding the polypeptides disclosed herein are also provided. An exemplary polynucleotide sequence encoding SEQ ID NO: 1 is shown below:

(SEQ ID NO: 2)
atggacataa agatagatat tagtatttct ggtgataaat
ttacggtgac tactaggagg gaaaatgaag aaagaaaaaa
atatctacct ctccaaaaag aaaaaactac tgatgttatc
aaacctgatt atcttgagta cgatgacttg ttagatagag
atgagatgtt tactattcta gaggaatatt ttatgtacag
aggtctatta ggcctcagaa taaaatatgg acgactcttt
aacgaaatta aaaaattcga caatgatgcg gaagaacaat
tcggtactat agaagaactc aagcagaaac ttagattaaa
ttctgaagag ggagcagata actttataga ttatataaag
gtacaaaaac aggatatcgt caaacttact gtatacgatt
gcatatctat gataggattg tgtgcatgcg tggtagatgt
ttggagaaat gagaaactgt tttctagatg gaaatattgt
ttacgagcta ttaaactgtt tattaatgat cacatgcttg
ataagataaa atctatactg cagaatagac tagtgtatgt
ggaaatgtca tag

These polynucleotides include DNA, cDNA and RNA sequences which encode the polypeptide of interest. Silent mutations in the coding sequence result from the degeneracy (i.e., redundancy) of the genetic code, whereby more than one codon can encode the same amino acid residue. Thus, for example, leucine can be encoded by CTT, CTC, CTA, CTG, TTA, or TTG; serine can be encoded by TCT, TCC, TCA, TCG, AGT, or AGC; asparagine can be encoded by AAT or AAC; aspartic acid can be encoded by GAT or GAC; cysteine can be encoded by TGT or TGC; alanine can be encoded by GCT, GCC, GCA, or GCG; glutamine can be encoded by CAA or CAG; tyrosine can be encoded by TAT or TAC; and isoleucine can be encoded by ATT, ATC, or ATA. Tables showing the standard genetic code can be found in various sources (see, for example, L. Stryer, 1988, Biochemistry, 3.sup.rd Edition, W.H. 5 Freeman and Co., NY).

A nucleic acid encoding a polypeptide can be cloned or amplified by in vitro methods, such as the polymerase chain reaction (PCR), the ligase chain reaction (LCR), the transcription-based amplification system (TAS), the self-sustained sequence replication system (3SR) and the Qβ replicase amplification system (QB). For example, a polynucleotide encoding the protein can be isolated by polymerase chain reaction of cDNA using primers based on the DNA sequence of the molecule. A wide variety of cloning and in vitro amplification methodologies are well known to persons skilled in the art. PCR methods are described in, for example, U.S. Pat. No. 4,683,195; Mullis et al., Cold Spring Harbor Symp. Quant. Biol. 51:263, 1987; and Erlich, ed., PCR Technology, (Stockton Press, NY, 1989). Polynucleotides also can be isolated by screening genomic or cDNA libraries with probes selected from the sequences of the desired polynucleotide under stringent hybridization conditions.

The polynucleotides encoding the disclosed polypeptide include a recombinant DNA which is incorporated into a vector into an autonomously replicating plasmid or virus or into the genomic DNA of a prokaryote or eukaryote, or which exists as a separate molecule (such as a cDNA) independent of other sequences. The nucleotides of the invention can be ribonucleotides, deoxyribonucleotides, or modified forms of either nucleotide. The term includes single and double forms of DNA.

Plasmids for expression in bacteria are well known in the art, and include pBR322 based plasmids. As noted above, a polynucleotide sequence encoding a polypeptide can be operatively linked to expression control sequences. An expression control sequence operatively linked to a coding sequence is ligated such that expression of the coding sequence is achieved under conditions compatible with the expression control sequences. The expression control sequences include, but are not limited to, appropriate promoters, enhancers, transcription terminators, a start codon (i.e., ATG) in front of a protein-encoding gene, splicing signal for introns, maintenance of the correct reading frame of that gene to permit proper translation of mRNA, and stop codons.

Vectors can also be used for expression in yeast such as S. cerevisiae or Kluyveromyces lactis. Several promoters are known to be of use in yeast expression systems such as the constitutive promoters plasma membrane H⁺-ATPase (PMA1), glyceraldehyde-3-phosphate dehydrogenase (GPD), phosphoglycerate kinase-1 (PGK1), alcohol dehydrogenase-1 (ADH1), and pleiotropic drug-resistant pump (PDR5). In addition, many inducible promoters are of use, such as GAL1-10 (induced by galactose), PHO5 (induced by low extracellular inorganic phosphate), and tandem heat shock HSE elements (induced by temperature elevation to 37° C.). Promoters that direct variable expression in response to a titratable inducer include the methionine-responsive MET3 and MET25 promoters and copper-dependent CUP1 promoters. Any of these promoters may be cloned into multicopy (2μ) or single copy (CEN) plasmids to give an additional level of control in expression level. The plasmids can include nutritional markers (such as URA3, ADE3, HIS1, and others) for selection in yeast and antibiotic resistance (AMP) for propagation in bacteria. Plasmids for expression on K. lactis are known, such as pKLAC1. Thus, in one example, after amplification in bacteria, plasmids can be introduced into the corresponding yeast auxotrophs by methods similar to bacterial transformation.

The polypeptides can be expressed in a variety of yeast strains. For example, seven pleiotropic drug-resistant transporters, YOR1, SNQ2, PDR5, YCF1, PDR10, PDR11, and PDR15, together with their activating transcription factors, PDR1 and PDR3, have been simultaneously deleted in yeast host cells, rendering the resultant strain sensitive to drugs. Yeast strains with altered lipid composition of the plasma membrane, such as the erg6 mutant defective in ergosterol biosynthesis, can also be utilized. Proteins that are highly sensitive to proteolysis can be expressed in a yeast lacking the master vacuolar endopeptidase Pep4, which controls the activation of other vacuolar hydrolases. Heterologous expression in strains carrying temperature-sensitive (ts) alleles of genes can be employed if the corresponding null mutant is inviable.

Viral vectors can also be prepared encoding the polypeptides disclosed herein. A number of viral vectors have been constructed, including polyoma, SV40 (Madzak et al., 1992, J. Gen. Virol., 73:15331536), adenovirus (Berkner, 1992, Cur. Top. Microbiol. Immunol., 158:39-6; Berliner et al., 1988, Bio Techniques, 6:616-629; Gorziglia et al., 1992, J. Virol., 66:4407-4412; Quantin et al., 1992, Proc. Nad. Acad. Sci. USA, 89:2581-2584; Rosenfeld et al., 1992, Cell, 68:143-155; Wilkinson et al., 1992, Nucl. Acids Res., 20:2233-2239; Stratford-Perricaudet et al., 1990, Hum. Gene Ther., 1:241-256), vaccinia virus (Mackett et al., 1992, Biotechnology, 24:495-499), adeno-associated virus (Muzyczka, 1992, Curr. Top. Microbiol. Immunol., 158:91-123; On et al., 1990, Gene, 89:279-282), herpes viruses including HSV and EBV (Margolskee, 1992, Curr. Top. Microbiol. Immunol., 158:67-90; Johnson et al., 1992, J. Virol., 66:29522965; Fink et al., 1992, Hum. Gene Ther. 3:11-19; Breakfield et al., 1987, Mol. Neurobiol., 1:337-371; Fresse et al., 1990, Biochem. Pharmacol., 40:2189-2199), Sindbis viruses (H. Herweijer et al., 1995, Human Gene Therapy 6:1161-1167; U.S. Pat. Nos. 5,091,309 and 5,2217,879), alphaviruses (S. Schlesinger, 1993, Trends Biotechnol. 11:18-22; I. Frolov et al., 1996, Proc. Natl. Acad. Sci. USA 93:11371-11377) and retroviruses of avian (Brandyopadhyay et al., 1984, Mol. Cell. Biol., 4:749-754; Petropouplos et al., 1992, J. Virol., 66:3391-3397), murine (Miller, 1992, Curr. Top. Microbiol. Immunol., 158:1-24; Miller et al., 1985, Mol. Cell. Biol., 5:431-437; Sorge et al., 1984, Mol. Cell. Biol., 4:1730-1737; Mann et al., 1985, J. Virol., 54:401-407), and human origin (Page et al., 1990, J. Virol., 64:5370-5276; Buchschalcher et al., 1992, J. Virol., 66:2731-2739). Baculovirus (Autographa californica multinuclear polyhedrosis virus; AcMNPV) vectors are also known in the art, and may be obtained from commercial sources (such as PharMingen, San Diego, Calif.; Protein Sciences Corp., Meriden, Conn.; Stratagene, La Jolla, Calif.).

Thus, in one embodiment, the polynucleotide encoding one or more of the disclosed polypeptides is included in a viral vector. Suitable vectors include retrovirus vectors, orthopox vectors, avipox vectors, fowlpox vectors, capripox vectors, suipox vectors, adenoviral vectors, herpes virus vectors, alpha virus vectors, baculovirus vectors, Sindbis virus vectors, vaccinia virus vectors and poliovirus vectors. Specific exemplary vectors are poxvirus vectors such as vaccinia virus, fowlpox virus and a highly attenuated vaccinia virus (MVA), adenovirus, baculovirus and the like.

DNA sequences encoding a polypeptide can be expressed in vitro by DNA transfer into a suitable host cell. The cell may be prokaryotic or eukaryotic. The term also includes any progeny of the subject host cell. It is understood that all progeny may not be identical to the parental cell since there may be mutations that occur during replication. Methods of stable transfer, meaning that the foreign DNA is continuously maintained in the host, are known in the art.

Hosts cells can include microbial, yeast, insect and mammalian host cells. Methods of expressing DNA sequences having eukaryotic or viral sequences in prokaryotes are well known in the art. Non-limiting examples of suitable host cells include bacteria, archea, insect, fungi (for example, yeast), plant, and animal cells (for example, mammalian cells, such as human). Exemplary cells of use include Escherichia coli, Bacillus subtilis, Saccharomyces cerevisiae, Salmonella typhimurium, SF9 cells, C129 cells, 293 cells, Neurospora, and immortalized mammalian myeloid and lymphoid cell lines. Techniques for the propagation of mammalian cells in culture are well-known (see, Jakoby and Pastan (eds), 1979, Cell Culture. Methods in Enzymology, volume 58, Academic Press, Inc., Harcourt Brace Jovanovich, N.Y.). Examples of commonly used mammalian host cell lines are VERO and HeLa cells, CHO cells, and WI38, BHK, and COS cell lines, although cell lines may be used, such as cells designed to provide higher expression desirable glycosylation patterns, or other features. As discussed above, techniques for the transformation of yeast cells, such as polyethylene glycol transformation, protoplast transformation and gene guns are also known in the art (see Gietz and Woods Methods in Enzymology 350: 87-96, 2002).

Transformation of a host cell with recombinant DNA can be carried out by conventional techniques as are well known to those skilled in the art. Where the host is prokaryotic, such as, but not limited to, E. coli, competent cells which are capable of DNA uptake can be prepared from cells harvested after exponential growth phase and subsequently treated by the CaCl₂ method using procedures well known in the art. Alternatively, MgCl₂ or RbCl can be used. Transformation can also be performed after forming a protoplast of the host cell if desired, or by electroporation.

When the host is a eukaryote, such methods of transfection of DNA as calcium phosphate coprecipitates, conventional mechanical procedures such as microinjection, electroporation, insertion of a plasmid encased in liposomes, or virus vectors can be used. Eukaryotic cells can also be co-transformed with polynucleotide sequences encoding a polypeptide of interest, and a second foreign DNA molecule encoding a selectable phenotype, such as the herpes simplex thymidine kinase gene. Another method is to use a eukaryotic viral vector, such as simian virus 40 (SV40) or bovine papilloma virus, to transiently infect or transform eukaryotic cells and express the protein (see for example, Eukaryotic Viral Vectors, Cold Spring Harbor Laboratory, Gluzman ed., 1982).

### Methods of Treatment of a Subject with Septic Shock

Methods are provided herein for treating a subject with septic shock, or for preventing septic shock in a subject. The subject can be any mammalian subject, including veterinary and human subjects. Any of the polypeptides disclosed herein can be used in these methods.

In one embodiment, a method is provided for treating a subject with septic shock, wherein the method includes selecting a subject with septic shock; and administering to the subject a therapeutically effective amount of a therapeutic polypeptide, wherein the therapeutic polypeptide comprises or consists of: (a) a first polypeptide consisting of 11 to 18 consecutive amino acids of the amino acid sequence set forth as SEQ ID NO: 1 (A52R), or wherein the first polypeptide consists of 8 to 11 amino acids of the amino acid sequence set forth as SEQ ID NO: 1 with a single amino acid substitution thereof and the first polypeptide covalently binds to toll-like receptor 3 with the same or greater affinity as the 11 to 18 consecutive amino acids of the amino acid sequence set forth as SEQ ID NO: 1, and (b) a second peptide consisting of seven to ten consecutive arginine residues, wherein the second polypeptide is covalently bound to the first polypeptide, such as in a fusion polypeptide.

In a further non-limiting example, the polypeptide consists of, or consists essentially of, amino acids 70 to 86 of SEQ ID NO: 1 covalently linked to 9 arginine residues. In a further non-limiting example, the polypeptide consists of, or consists essentially of, amino acids 70 to 86 of SEQ ID NO: 1 with a single amino acid substitution that retains the ability to inhibit TLR activity covalently linked to 9 arginine residues. Additional polypeptides consist of, or consist essentially of, amino acids 125 to 135 of SEQ ID NO: 1, or a single substitution thereof that retains the ability to inhibit TLR activity, covalently linked to 9 arginine residues.

Septic shock can be caused by infection with a gram negative or gram positive bacteria. In one embodiment the subject has septic shock, diagnosed by the presence of one or both of the following: (1) evidence of infection, through a positive blood culture (2) refractive hypotension (despite adequate fluid resuscitation) which in adults is diagnosed as a systolic blood pressure of less than about 90 mmHg, or a mean arterial pressure (MAP) of less than about 60 mmHg, or a reduction of 40 mmHg in the systolic blood pressure from baseline, while in children it is a blood pressure of less than two standard deviations (SD) of the normal blood pressure. In addition to these two criteria above, the subject can have two or more of the following: (a) heart rate of greater than about 90 beats per minute; (b) body temperature of less than about 36 or greater than about 38° C.; (3) hyperventilation (high respiratory rate) greater than 20 breaths per minute or, on blood gas, a PₐCO₂ less than about 32 mmHg; (4) white blood cell count less than 4000 cells/mm³ or greater than about 12000 cells/mm³ (<4×10⁹ or >12×10⁹ cells/L). In one example, a therapeutically effective amount of the compound is that which provides either subjective relief of a symptom(s) or an objectively identifiable improvement as noted by the clinician or other qualified observer in at least one of the parameters described above.

Methods are provided herein for treating a subject with septic shock, such as septic shock resulting from an infection with gram negative bacteria. In several examples, the gram negative bacteria is Escherichia coli, Klebsiella pneumoniae, Proteus species, Pseudomonas aeruginosa or Salmonella typhimurium. Methods are also provided herein for treating a subject with septic shock resulting from an infection with gram positive bacteria. In several examples, gram positive bacteria is a species of Staphlococci, Streptococci or Pneumococci. Thus, methods are provided herein for the treatment of septic shock caused by either a gram negative or gram positive bacteria. Methods are also provided herein for the treatment of septic shock caused by a fungus.

A polypeptide can be administered by any means known to one of skill in the art (see Banga, A., “Parenteral Controlled Delivery of Therapeutic Peptides and Proteins,” in Therapeutic Peptides and Proteins, Technomic Publishing Co., Inc., Lancaster, Pa., 1995) either locally or systemically, such as by intramuscular, subcutaneous, intraperitoneal or intravenous injection, but even oral, nasal, transdermal or anal administration is contemplated. In one embodiment, administration is by subcutaneous or intramuscular injection. To extend the time during which the peptide or protein is available, the peptide or protein can be provided as an implant, an oily injection, or as a particulate system. The particulate system can be a microparticle, a microcapsule, a microsphere, a nanocapsule, or similar particle. A particulate carrier based on a synthetic polymer has been shown to act as an adjuvant to enhance the immune response, in addition to providing a controlled release.

Controlled release parenteral formulations can be made as implants, oily injections, or as particulate systems. For a broad overview of protein delivery systems, see Banga, Therapeutic Peptides and Proteins: Formulation, Processing, and Delivery Systems, Technomic Publishing Company, Inc., Lancaster, Pa., 1995. Particulate systems include microspheres, microparticles, microcapsules, nanocapsules, nanospheres, and nanoparticles. Microcapsules contain the therapeutic protein as a central core. In microspheres, the therapeutic agent is dispersed throughout the particle. Particles, microspheres, and microcapsules smaller than about 1 μm are generally referred to as nanoparticles, nanospheres, and nanocapsules, respectively. Capillaries have a diameter of approximately 5 μm so that only nanoparticles are administered intravenously. Microparticles are typically around 100 μm in diameter and are administered subcutaneously or intramuscularly (see Kreuter, Colloidal Drug Delivery Systems, J. Kreuter, ed., Marcel Dekker, Inc., New York, N.Y., pp. 219-342, 1994; Tice & Tabibi, Treatise on Controlled Drug Delivery, A. Kydonieus, ed., Marcel Dekker, Inc. New York, N.Y., pp. 315-339, 1992).

Polymers can be used for ion-controlled release. Various degradable and nondegradable polymeric matrices for use in controlled drug delivery are known in the art (Langer, Accounts Chem. Res. 26:537, 1993). For example, the block copolymer, polaxamer 407 exists as a viscous yet mobile liquid at low temperatures but forms a semisolid gel at body temperature. It has shown to be an effective vehicle for formulation and sustained delivery of recombinant interleukin-2 and urease (Johnston et al., Pharm. Res. 9:425, 1992; and Pec, J. Parent. Sci. Tech. 44(2):58, 1990). Alternatively, hydroxyapatite has been used as a microcarrier for controlled release of proteins (Ijntema et al., Int. J. Pharm. 112:215, 1994). In yet another aspect, liposomes are used for controlled release as well as drug targeting of the lipid-capsulated drug (Betageri et al., Liposome Drug Delivery Systems, Technomic Publishing Co., Inc., Lancaster, Pa., 1993). Numerous additional systems for controlled delivery of therapeutic proteins are known (e.g., U.S. Pat. No. 5,055,303; U.S. Pat. No. 5,188,837; U.S. Pat. No. 4,235,871; U.S. Pat. No. 4,501,728; U.S. Pat. No. 4,837,028; U.S. Pat. No. 4,957,735; and U.S. Pat. No. 5,019,369; U.S. Pat. No. 5,055,303; U.S. Pat. No. 5,514,670; U.S. Pat. No. 5,413,797; U.S. Pat. No. 5,268,164; U.S. Pat. No. 5,004,697; U.S. Pat. No. 4,902,505; U.S. Pat. No. 5,506,206; U.S. Pat. No. 5,271,961; U.S. Pat. No. 5,254,342; and U.S. Pat. No. 5,534,496).

In one specific, non-limiting example, a pharmaceutical composition for intravenous administration would include about 0.1 μg to 10 mg of immunogenic polypeptide per patient per day. Dosages from 0.1 up to about 100 mg per subject per day can be used, particularly if the agent is administered to a body cavity or into a lumen of an organ. Actual methods for preparing administrable compositions will be known or apparent to those skilled in the art and are described in more detail in such publications as Remingtons Pharmaceutical Sciences, 19^{th} Ed., Mack Publishing Company, Easton, Pa., 1995.

Single or multiple administrations of the compositions are administered depending on the dosage and frequency as required and tolerated by the subject. In one embodiment, the dosage is administered once as a bolus, but in another embodiment can be applied periodically until a therapeutic result is achieved. Generally, the dose is sufficient to treat or ameliorate symptoms or signs of disease without producing unacceptable toxicity to the subject. Systemic or local administration can be utilized.

In another embodiment, a pharmaceutical composition includes a nucleic acid encoding one or more of the polypeptides disclosed herein. A therapeutically effective amount of the polynucleotide can be administered to a subject, such as a subject with septic shock. In one specific, non-limiting example, a therapeutically effective amount of the polynucleotide is administered to a subject to treat septic shock induced by a gram negative bacteria. In another specific, non-limiting example, a therapeutically effective amount of the polynucleotide is administered to a subject to treat septic shock induced by a gram positive bacteria.

One approach to administration of nucleic acids is direct immunization with plasmid DNA, such as with a mammalian expression plasmid. As described above, the nucleotide sequence encoding a polypeptide can be placed under the control of a promoter to increase expression of the molecule.

Administration of nucleic acid constructs is well known in the art and taught, for example, in U.S. Pat. No. 5,643,578; U.S. Pat. No. 5,593,972 and U.S. Pat. No. 5,817,637; and U.S. Pat. No. 5,880,103. The methods include liposomal delivery of the nucleic acids (or of the synthetic peptides themselves).

In another approach to using nucleic acids for immunization, a polypeptide can also be expressed by attenuated viral hosts or vectors or bacterial vectors. Recombinant vaccinia virus, adeno-associated virus (AAV), herpes virus, retrovirus, or other viral vectors can be used to express the peptide or protein. For example, vaccinia vectors and methods of administration are described in U.S. Pat. No. 4,722,848. BCG (Bacillus Calmette Guerin) provides another vector for expression of the peptides (see Stover, Nature 351:456-460, 1991).

When a viral vector is utilized, it is desirable to provide the recipient with a dosage of each recombinant virus in the composition in the range of from about 10⁵ to about 10¹⁰ plaque forming units/mg mammal, although a lower or higher dose can be administered. The composition of recombinant viral vectors can be introduced into a subject with septic shock. Examples of methods for administering the composition into mammals include, but are not limited to, intravenous, subcutaneous, intradermal or intramuscular administration of the nucleic acid, such as virus or other vector including the nucleic acid encoding the disclosed polypeptides. Generally, the quantity of recombinant viral vector, carrying the nucleic acid sequence of a polypeptide to be administered is based on the titer of virus particles. An exemplary range of the virus to be administered is 10⁵ to 10¹⁰ virus particles per mammal, such as a human.

In additional methods, the subject is also administered an additional agent, such as anti-microbial agent or a corticosteroid. In further methods, the subject is also administered activate protein C and/or intensive fluid resuscitation. In several examples, this administration is sequential. In other examples, this administration is simultaneous.

Suitable anti-microbial agents include any antibiotic, which includes any compound that decreases or abolishes the growth of a pathogen, such as a gram negative bacteria, gram positive bacterial, fungus or protozoa. These compounds include the following: amino glycosides such as amikacin, neomycin, streptomycin or gentamycin, ansamycins such as geldanamycin or herbamycin, a carbacephem such as loracarbef, a carbapenem such as meropenem, a cephalosporin (including first, second, third and forth generation cepalosporins) such as cefazolin or cefepine, a glycopeptides such as vancomycina macrolide such as azithromycin, a penicillin such as ampicillin or amoxicillin, a polypeptide such as bacitracin, a quinolone such as ciprofloxacin, a sulfonamide such as mafenide, a tetracycline such as doxyclycine.

The disclosure is illustrated by the following non-limiting Examples.

### EXAMPLES

Described herein are experiments demonstrating that A52R polypeptides can be used for the treatment of septic shock, such as the septic shock induced by gram negative bacteria.

### Example 1

### Materials and Methods

Animals. Male C57BL/6 mice (8-12 weeks old) or Female BALB/c mice (8-12 weeks old) were purchased from the Jackson Laboratory (Bar Harbor, Me.). All animals were maintained in a laminar-flow, specific pathogen-free atmosphere at

Reagents. Peptides were synthesized commercially, with the following sequences: P13: (DIVKLTVYDCI-RRRRRRRRR) (SEQ ID NO: 3); and scrambled control: (ITCVDVDLIYK-RRRRRRRRR) (SEQ ID NO: 4). Lipopolysaccharide purified from E. coli was obtained from SIGMA.

Quantification of cytokines from cell supernatants. IL-6 and MIP-2 ELISA kits were used to assay cytokine levels in cell culture supernatants according to the protocols provided by the manufacturer (R&D Systems, Minneapolis, Minn.).

Serum cytokines and soluble ICAM-1. BALB/c female mice were injected intraperitoneally (i.p.) with 5 mg/kg of LPS and peptide P13 (15, 50, or 75 μg) or PBS was injected i.p. 30 minutes later. Two hours after injection of LPS, serum was collected and TNF-α and soluble ICAM-1 quantified by ELISA (R&D Systems).

LPS-induced sepsis. Female BALB/c mice were injected i.p. with 70 mg/kg of LPS. At 2 and 6 hours after injection of LPS, mice were treated with scrambled control peptide or peptide P13. The peptides were administered subcutaneously at 75 μg/animal/injection time, with 10 mice/treatment group. The mice were monitored every 3-4 hours and the experiment was terminated after 96 hours.

Statistical analysis. All group comparisons were performed using Student's t test or ANOVA. Differences were considered significant at p<0.05

### Example 3

### P13 Peptide Inhibits Cytokine Production by Endothelial Cells in Response to LPS

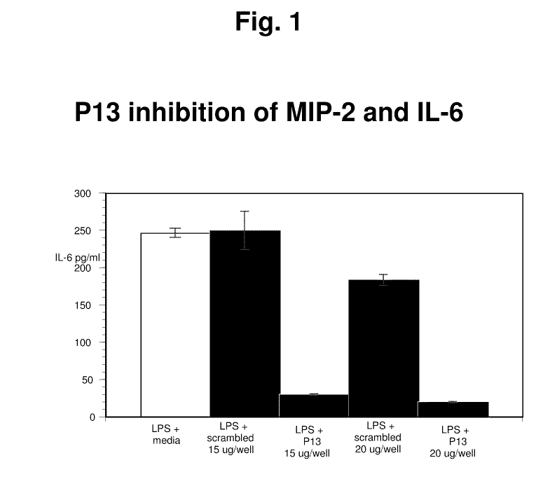

Because endothelial cells can be activated and even damaged in response to LPS exposure, the effect of treatment with peptide P13 on LPS-induced cytokine responses from the murine bEND.3 endothelial cell line was examined. Treatment of the bEND cells with LPS induced secretion of MIP-2 and IL-6. Treatment with 15 μg/well or 20 μg/well of peptide P13 resulted in a dramatic inhibition (>90%) of both MIP-2 and IL-6 production (FIG. 1). Treatment with matching concentrations of the scrambled control peptide showed only modest inhibition. These results demonstrate the ability of P13 peptide to inhibit cytokine production by endothelial cells in response to LPS.

### Example 3

### P13 Peptide Inhibits LPS-Induced In Vivo Cytokine and Soluble ICAM-1 Production

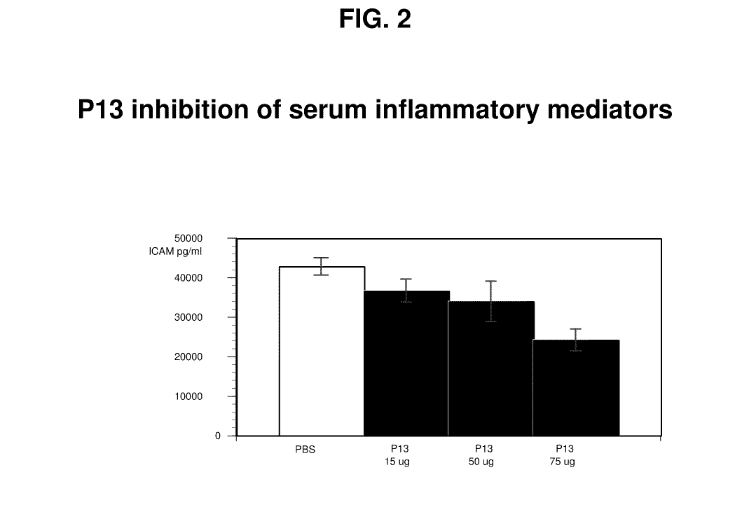

To determine the effect of peptide treatment on the induction of serum inflammatory mediators, mice were injected with a sub-lethal high dose of LPS and 30 minutes later with P13 or phosphate buffered saline (PBS). Serum was collected 2 hours after treatment with LPS. TNF-α and soluble ICAM-1 were quantified by ELISA. Treatment of mice with 75 μg of P13 resulted in a significant inhibition of TNF-α, whereas treatment with 50 μg did not reduce TNF-α production (FIG. 2). The effect of delayed peptide P13 treatment at different doses of peptide on LPS-induced production of soluble ICAM-1 was examined. Treatment with peptide P13 showed a dose-dependent inhibition of soluble ICAM-1, with each dose of peptide P13 giving a statistically significant inhibition of ICAM-1 (FIG. 2).

### Example 4

### P13 Peptide is Protective In Vivo in an LPS-Induced Sepsis Model

The data presented herein demonstrate the effectiveness of peptide P13 to enhance survival in animals treated with low dose LPS plus D-GaLN and in animals receiving high dose LPS alone. Administration of P13 either i.p. or subcutaneously were both effective, as was delaying peptide administration after administration of LPS.

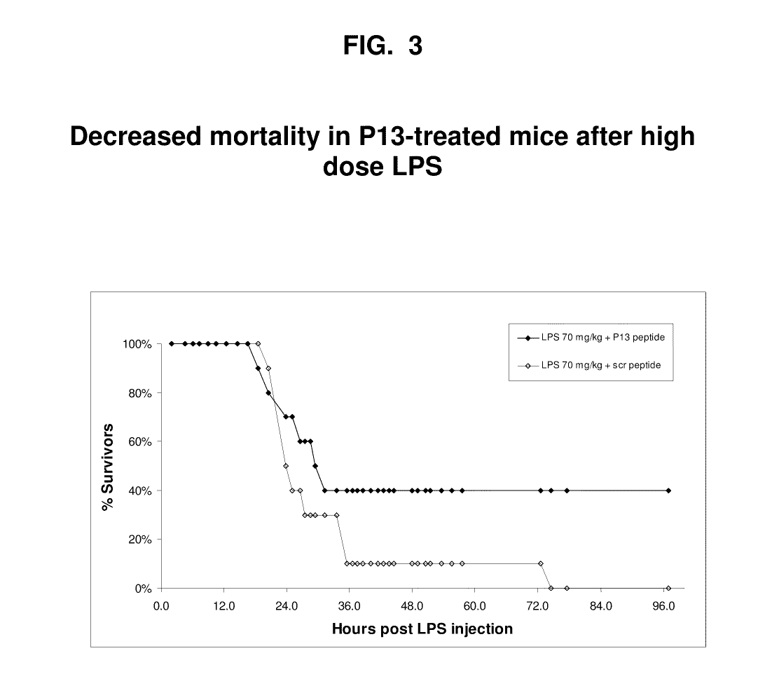

As P13 inhibited production of inflammatory mediators in vivo, an LPS-induced murine sepsis model was used to determine the effect of peptide P13 on survival. Mice were injected with lethal dose of LPS (70 mg/kg) and treated 2 and 6 hours later with scrambled control peptide (75 μg) or a matching concentration of peptide P13. Mice treated with scrambled peptide demonstrated 90% lethality at 36 hours and 100% lethality at 74 hours (FIG. 3). Treatment of mice with LPS and peptide P13 resulted in a reduction of lethality, with 60% lethality at 36 and 74 hours. The experiment was terminated at 96 hours and P13 treated mice remained at 40% survival (FIG. 3). These studies confirm the in vivo activity of peptide P13 to reduce lethality in an LPS sepsis model even when peptide treatment is initiated after administration of LPS.

TLR4 plays a central role in recognizing LPS and initiating immune cell signaling for the activation of inflammatory pathways. P13 is capable of in vitro inhibition of TLR-dependent signaling (McCoy et al., J. Immun. 174:3006-3014, 2005). The results described above demonstrate that P13 peptide is protective against LPS-induced inflammation and resultant tissue damage and organ failure. Without being bound by theory, the he major findings are: i) peptide P13 reduced the production of inflammatory mediators in HC—NPC co-cultures and cultured endothelial cells stimulated with LPS; and ii) treatment of mice with high dose LPS and peptide P13 resulted in decreased serum TNF-α production and reduced ICAM-1 levels and this inhibition of serum inflammatory mediators correlated with improved survival compared to mice treated with LPS and control scrambled peptide.

Sepsis resulting from gram-negative bacterial infection continues to be a major cause of morbidity and mortality and is associated with production of proinflammatory cytokines, activation of cell adhesion molecules, and induction of cell apoptosis, all of which contribute to multi-organ injury (Brown and Jones, Front. Biosci. 9:1201-1217, 2004). Joshi and colleagues (Joshi et al., FEBS Lett. 555:180-184, 2003) have suggested that the multi-organ involvement and death associated with severe sepsis is the result of the simultaneous activation of both an inflammatory response and cell apoptosis. The inflammatory response seen in sepsis is initiated by exposure of the host innate immune system to either bacteria or bacterial products (such as LPS). LPS activates the TLR signaling response which culminates in NF-κB activation, initiating an innate immune response characterized by release of proinflammatory mediators. If this response is not appropriately regulated, the inflammatory response continues and contributes to tissue destruction and resultant organ failure. Consistent with this observation are reports of increased cytokines, such as TNF-α and IL-I, in the serum of patients with sepsis. In addition, extensive apoptoic cell death was reported in both patients and animal models of sepsis (Hotchkiss and Karl, N. Engl. J. Med. 348:138-150, 2003, Mathiak et al., Br. J. Pharmacol. 131:383-386, 2000; Grobmyer et al., Mol. Med. 5:585-594, 1999). Endothelial cell apoptosis has been seen in disseminated intravascular coagulation, systemic vascular collapse, multiorgan failure and acute respiratory distress syndrome, all complications arising from sepsis (Bannerman and Goldblum, Am. J. Physiol. Lung Cell Mol. Physiol. 284:L899-914, 2003).

The liver is believed to integrate the inflammatory response during gram negative infections through both the clearance of microbes and their products and through the production of inflammatory mediators and acute phase proteins (Johnson and Billiar, World J Surg 22:187-196, 1998; Pastor and Billiar, New Horiz 3:65-72, 1995). It has been shown that the liver is the main organ involved in the clearance of LPS from the bloodstream and also plays a critical role in the identification and processing of LPS (Su et al., Am J Physiol Gastrointest Liver Physiol Sep; 283(3):G640-G645, 2002; Mathison et al., J Immunol 123:2133-2143, 1979). Kupffer cells and other immune cells in the liver are thought to be a major source for the production of inflammatory cytokines that further contribute to the inflammatory state during sepsis (Curran et al., J Exp Med 170:1769-1774, 1989; West et al., Infect Immun 49:563-570, 1985). The distinct subtypes of liver cells are arrayed in close proximity to each other for the coordinated production of inflammatory mediators in response to LPS (Bhatia et al., FASEB J 13:1883-1900, 1999).

The liver consists of parenchymal cells (hepatocytes) and nonparenchymal cells (NPC), such as Kupffer cells, sinusoidal endothelial cells, hepatic stellate cells, and dendritic cells. TLR4 is present on hepatocytes and NPCs and these cell populations all possess intact TLR4 signaling pathways (Liu et al., Infect Immun 70:3433-42, 2002; Su et al., Hepatology 31(4):932-936, 2000). Two main signaling pathways have been identified in association with TLR4. One is an early response involving the adaptor proteins MyD88 and Mal (TRAP) that leads to rapid activation of mitogen-activated protein kinases (MAPK), and thereby activation of the transcription factor NF-κB with subsequent production of pro-inflammatory responses (see, for example, Fitzgerald et al., Nature 413:78-83, 2001). A later response that is not MyD88-dependent involves the adaptor proteins TRIF and TRAM, which in turn activate IRF3 and stimulate the production of type I interferons (see, for example, Fitzgerald et al., Microbes Infect 6:1361-1367, 2004). It is possible that these separate activation pathways may differentiate LPS-signaling and LPS-clearance.

Recently, only one agent, recombinant activated protein C, has achieved regulatory approval for the treatment of septic shock. The use of intensive fluid resuscitation has been shown to modify disease development, resulting in a significant reduction in mortality (Marshall et al., Nat Rev Drug Discov 2:391-405, 2003) and could be used in combination with any of the polypeptides disclosed herein, including, but not limited to, P13. Peptide P13 has previously been shown to inhibit production of proinflammatory mediators induced by a variety of both bacterial and viral PAMPs (McCoy et al., J. Immun. 174:3006-3014, 2005). Inhibition of multiple TLR-dependent responses, by targeting a common signaling component, could be an effective approach to controlling an inflammatory response.

It will be apparent that the precise details of the methods or compositions described may be varied or modified without departing from the spirit of the described invention. We claim all such modifications and variations that fall within the scope and spirit of the claims below.

1. A method for treating a subject with septic shock, comprising
selecting a subject with septic shock; and
administering to the subject a therapeutically effective amount of a therapeutic polypeptide, wherein the therapeutic polypeptide consists of:
(a) a first polypeptide consisting of 11 to 18 consecutive amino acids of the amino acid sequence set forth in SEQ ID NO: 1 (A52R), or wherein the first polypeptide consists of 11 to 18 consecutive amino acids of amino acid sequence set forth as SEQ ID NO: 1 with a single amino acid substitution thereof, wherein the first retains the ability to inhibit Toll-like receptor activity as the 11 to 18 consecutive amino acids of the amino acid sequence set forth in SEQ ID NO: 1, and
(b) a second polypeptide consisting of seven to ten consecutive arginine residues,
thereby treating the septic shock in the subject.

2. The method of claim 1, wherein the first polypeptide consists of ten to twelve consecutive amino acids of the amino acid sequence set forth in SEQ ID NO: 1.

3. The method of claim 1, wherein the first polypeptide consists of twelve amino acids of the sequence set forth in SEQ ID NO: 1.

4. The method of claim 1, wherein second polypeptide consists of nine arginine residues.

5. The method of claim 1, wherein the first polypeptide consists of the amino acid sequence set forth as amino acids 70-86 of SEQ ID NO: 1.

6. The method of claim 1, wherein the first polypeptide consists of the amino acid sequence set forth as amino acids 70 to 86 of SEQ ID NO: 1 with a single amino acid substitution, wherein the first polypeptide retains the ability to inhibit Toll-like receptor activity as a polypeptide comprising amino acids 70-86 of SEQ ID NO: 1.

7. The method of claim 1, wherein the therapeutic polypeptide consists of amino acids 125-135 of SEQ ID NO: 1 or a single amino acid substitution thereof that retains the ability to inhibit Toll-like receptor activity.

8. The method of claim 1, wherein the subject is infected with a gram negative bacteria.

9. The method of claim 8, wherein the gram negative bacteria is Escherichia coli, Klebsiella pneumoniae, Proteus species, Pseudomonas aeruginosa or Salmonella typhimurium.

10. The method of claim 1, wherein the subject is infected with a gram positive bacteria.

11. The method of claim 10, wherein the gram positive bacteria is a species of Staphlococci, Streptococci or Pneumococci.

12. The method of claim 1, wherein the subject is infected with a fungus.

13. The method of claim 1, further comprising administering to the subject a therapeutically effective amount of an anti-microbial agent.

14. The method of claim 1, further comprising administering to the subject a therapeutically effective amount of a corticosteroid.

15. The method of claim 1, wherein the first polypeptide consists of the amino acid sequence set forth as SEQ ID NO: 3, SEQ ID NO: 5, SEQ ID NO: 6, SEQ ID NO:7, SEQ ID NO:8, SEQ ID NO:9, SEQ ID NO: 10, SEQ ID NO:11, SEQ ID NO: 12, SEQ ID NO: 13, SEQ ID NO: 14, SEQ ID NO: 15, SEQ ID NO:16, SEQ ID NO: 17, SEQ ID NO:18, SEQ ID NO:19, SEQ ID NO: 20 or SEQ ID NO: 21.

16. The method of claim 15, wherein the subject is infected with a gram negative bacteria.

17. The method of claim 1, wherein the therapeutic polypeptide is administered orally.


SEQUENCE LISTING (22 sequences)

SEQ ID NO: 1 - protein, 190 aa, Vaccinia virus
MDIKIDISISGDKFTVTTRRENEERKKYLPLQKEKTTDVIKPDYLEYDDLLDRDEMFTILEEYFMYRGLLGLRIKYGRLFNEIKKFDNDAEEQFGTIEELKQKLRLNSEEGADNFIDYIKVQKQDIVKLTVYDCISMIGLCACVVDVWRNEKLFSRWKYCLRAIKLFINDHMLDKIKSILQNRLVYVEMS

SEQ ID NO: 2 - DNA, 573 bp, Vaccinia virus
atggacataaagatagatattagtatttctggtgataaatttacggtgactactaggagggaaaatgaagaaagaaaaaaatatctacctctccaaaaagaaaaaactactgatgttatcaaacctgattatcttgagtacgatgacttgttagatagagatgagatgtttactattctagaggaatattttatgtacagaggtctattaggcctcagaataaaatatggacgactctttaacgaaattaaaaaattcgacaatgatgcggaagaacaattcggtactatagaagaactcaagcagaaacttagattaaattctgaagagggagcagataactttatagattatataaaggtacaaaaacaggatatcgtcaaacttactgtatacgattgcatatctatgataggattgtgtgcatgcgtggtagatgtttggagaaatgagaaactgttttctagatggaaatattgtttacgagctattaaactgtttattaatgatcacatgcttgataagataaaatctatactgcagaatagactagtgtatgtggaaatgtcatag

SEQ ID NO: 3 - protein, 11 aa, Vaccinia virus
DIVKLTVYDCI

SEQ ID NO: 4 - protein, 20 aa, Vaccinia virus
ITCVDVDLIYKRRRRRRRRR

SEQ ID NO: 5 - protein, 17 aa, Vaccinia virus
EEYFMYRGLLGLRIKYG

SEQ ID NO: 6 - protein, 17 aa, Vaccinia virus
MDIKIDISISGDKFTVT

SEQ ID NO: 7 - protein, 18 aa, Vaccinia virus
GDKFTVTTRRENEERKKY

SEQ ID NO: 8 - protein, 17 aa, Vaccinia virus
NEERKKYLPLQKEKTTD

SEQ ID NO: 9 - protein, 16 aa, Vaccinia virus
QKEKTTDVIKPDYLEY

SEQ ID NO: 10 - protein, 17 aa, Vaccinia virus
KPDYLEYDDLLDRDEMF

SEQ ID NO: 11 - protein, 17 aa, Vaccinia virus
LDRDEMFTILEEYFMYR

SEQ ID NO: 12 - protein, 17 aa, Vaccinia virus
GLRIKYGRLFNEIKKFD

SEQ ID NO: 13 - protein, 17 aa, Vaccinia virus
NEIKKFDNDAEEQFGTI

SEQ ID NO: 14 - protein, 17 aa, Vaccinia virus
EEQFGTIEELKQKLRLN

SEQ ID NO: 15 - protein, 11 aa, Vaccinia virus
KLRLNSEEGAD

SEQ ID NO: 16 - protein, 11 aa, Vaccinia virus
NFIDYIKVQKQ

SEQ ID NO: 17 - protein, 11 aa, Vaccinia virus
SMIGLCACVVD

SEQ ID NO: 18 - protein, 11 aa, Vaccinia virus
VWRNEKLFSRW

SEQ ID NO: 19 - protein, 11 aa, Vaccinia virus
KYCLRAIKLFI

SEQ ID NO: 20 - protein, 11 aa, Vaccinia virus
NDHMLDKIKSI

SEQ ID NO: 21 - protein, 11 aa, Vaccinia virus
LQNRLVYVEMS

SEQ ID NO: 22 - DNA, 20 bp, Artificial Sequence
  synthetic CpG oligonucleotide
tccatgacgttcctgacgtt